(19) United States
(12) Patent Application Publication
(10) Pub. No.: US 2005/0203350 A1
(43) Pub. Date: Sep. 15, 2005

(54) Patient monitoring apparatus

(75) Inventor: Bernd Beck, Rangendingen (DE)

Correspondence Address: LERNER AND GREENBERG, PA, P O BOX 2480, HOLLYWOOD, FL, 33022-2480, US

(73) Assignee: Smiths Medical Deutschland GmbH

(21) Appl. No.: 11/064,338

(22) Filed: Feb. 23, 2005

(30) Foreign Application Priority Data
Mar. 10, 2004 (DE) 10 2004 012 042.0

(51) Int. Cl. A61B 5/00; A61B 5/02; A61B 5/08; A61B 10/00; G06F 17/00

(52) U.S. Cl. 600/300; 128/903; 128/920

(57) ABSTRACT

A patient monitoring apparatus includes at least one transducer for a patient, a transmission device for the patient and a reception device which is remote therefrom and to which an evaluation unit is connected. The transmission and reception devices each have at least one memory. The memory in the transmission unit stores an explicit or unique unalterable identification code. The reception unit has a keypad which can be used to input an identification code into its memory. A radio link between the transmission and reception units is set up only if the two stored identification codes are identical. If the radio link is interrupted for a prescribed period, the identification code in the reception unit is automatically erased, and a connection can be set up again only after the appropriate identification code has been input on the reception unit again. The radio link operates on the basis of the Bluetooth protocol.

### BACKGROUND OF THE INVENTION

Field of the Invention

The present invention relates to a patient monitoring apparatus having a transmission unit for a patient. The transmission unit transmits measured data to a reception unit over a radio link. At least one transducer for the patient is connected to the transmission unit.

An apparatus of that type is known from German Published, Non-Prosecuted Patent Application DE 198 51 276 A1, corresponding to U.S. Pat. No. 6,139,503. That document provides a plurality of pressure transducers, which can be fixed to a support, for invasively measuring a patient's blood pressure. Measured data from the pressure transducer are transmitted to a monitor from the support through the use of a single common signal transmission device. In that configuration, the signal transmission device may be a cable or a wireless signal transmission device which includes a transmitter on the support and on the receiver on the monitor.

Measuring apparatuses for monitoring patient data which transmit that data wirelessly to a signal receiver, such as a monitor, are also known from German Published, Non-Prosecuted Patent Application DE 100 09 591 A1; European Patent EP 0 672 381 B1, corresponding to U.S. Pat. No. 5,482,049; U.S. Patent Application Publication Nos. 2001/0047125 A1 and U.S. 2002/0013614 A1; International Publication No. WO 01/45014 A1, corresponding to U.S. Patent Application Publication Nos. 2004/0162466 A1, 2004/0030226 A1 and 2001/0047125 A1; International Publication No. WO 01/97907 A2, corresponding to U.S. Patent Application Publication No. 2002/0013614 A1; and International Publication No. WO 02/064032 A2, corresponding to U.S. Patent Application Publication No. 2002/0115914 A1, with some specifications involving the signals on the radio link being transmitted on the basis of the Bluetooth protocol.

The known Bluetooth protocol is suitable for short-range radio links up to approximately 10 m, normally operates in a frequency range from 2.402 GHz to 2.480 GHz, and performs a frequency change 1600 times per second in 79 steps at a respective interval of 1 MHz (“frequency hopping” method). Advantages of that method are, inter alia, low power consumption and secure transmission as a result of a lack of susceptibility to faults, which means that it is also possible to operate a plurality of appliances together in one room. In contrast to transmission using infrared signals, it is also not necessary to have a direct line of sight between transmitter and receiver. The very low transmission power means that there is also no risk of other electronic appliances being disturbed.

In medical technology and particularly in operating theaters and on intensive care wards, a large volume of patient data needs to be continually captured and transmitted to an evaluation unit, such as a monitor. That requires complex “cabling” which is not only complex to connect and to lay but also restricts surgeons and clinical staff in their freedom of movement, which entails the risk of cables being inadvertently disconnected, swapped or even broken and, furthermore, occasionally exhibiting signs of corrosion, which can result in dangerous situations for the patient. In that regard, radio transmission, above all using the Bluetooth method, affords significant advantages. Nevertheless, it is also necessary to solve specific problems in order to be able to carry out radio transmission using the Bluetooth protocol from a patient's measuring equipment to an evaluation unit in an operating theater or on an intensive care ward.

In that context, it should be taken into account that the patient's measuring equipment normally stays on the patient when he or she is brought from the operating theater, for example, to an observation station or to a hospital room, whereas the monitoring unit normally remains fixed in the respective room, that is to say the operating theater, the observation room or the patient's room. That mean that the transmitter and receiver need to be able to cooperate with various remote stations, but under no circumstances must there be any mix-ups.

In order to increase patient safety, it is also necessary for the state of the radio link to be continually monitored and signaled in the event of a connection being interrupted. The same applies to the monitoring of the patient's measuring equipment. Brief disturbances in the signal transmission must not have any noticeable effects. In addition, the overall system should be clear and easy to operate. Further requirements can be found in the description below.

### SUMMARY OF THE INVENTION

It is accordingly an object of the invention to provide a patient monitoring apparatus, which overcomes the hereinafore-mentioned disadvantages of the heretofore-known devices of this general type and which operates safely and without error even in particularly critical instances of use, such as in an operating theater, intensive care ward or observation ward.

With the foregoing and other objects in view there is provided, in accordance with the invention, a patient monitoring apparatus. The apparatus comprises a reception unit having an input device for inputting an identification code and a memory for storing the input identification code. A transmission unit for a patient stores a unique identification code and transmits measured data to the reception unit over a radio link. At least one transducer for the patient is connected to the transmission unit. The transmission unit and the reception unit have a connection set up therebetween only if the identification codes in the transmission unit and the reception unit are identical.

The basic principle underlying the invention is that each patient's transmission unit is allocated an explicit or unique individual identification code, and the reception unit, which is remote from the patient, picks up a radio link only when this identification code has been input into it. After a connection has been dropped, the identification code needs to be re-input on the reception unit that is remote from the patient. By way of example, a connection is also dropped when the patient's transmission unit is physically removed from the range of the radio link, which is on the order of magnitude of 10 m. Therefore, if the patient with the measuring equipment and the transmission unit is taken from the operating theater, for example, then the radio link is automatically dropped. If a new patient with corresponding measuring equipment and a transmission unit is then brought into the operating theater, a radio link to the fixed reception unit is not picked up until the new identification code for the new transmission unit has been input into the reception unit.

It is naturally also possible to terminate the radio link by actively operating a key on the transmission unit and/or on the reception unit.

In order to increase the security of transmission, the patient's measured data are buffer-stored in a buffer memory in the transmission unit and are transmitted as a data packet at predetermined intervals of time. Corresponding buffering may also be provided in the receiver. In the event of brief disturbances on the radio link, the receiver can request the transmission of an appropriate data packet. The data packet may be transmitted every 100 ms, for example, preferably in the form of “slip frames”, which are unpacked in the receiver and are buffer-stored in the data buffer.

Provision may also be made for one and the same data packet to be transmitted twice in succession and for the two successive data packets to be compared with one another in the receiver and to be considered valid only if they are identical. If they are not identical, then a new data transmission is requested. A resultant delay in the data transmission is on the order of magnitude of a few 100 ms (maximally 500 ms) and is uncritical for the patient data which are to be monitored. The power supply for the patient's equipment is provided by batteries or chargeable storage batteries due to the required mobility. The patient's transmission unit continually monitors the charge state of the batteries or storage batteries and indicates it on the transmission unit. In addition, these data are also sent to the reception unit, where they are likewise indicated.

The transmission power can be disconnected when there is no transmission taking place, in order to minimize the power consumption of the patient's transmission unit.

The invention achieves a large number of advantages, a brief summary of which includes the following:

The radio link in the operating theater avoids disruptive cables which restrict or disturb the working radius of the medical staff. The safety risk which the cables cause for patients and users through stumbling or getting caught does not arise. Functional faults on the cable or on plug connectors as a result of damage, incorrect fixing, incorrect handling, wear or corrosion are avoided. A tangle of cables with the risk of mix-ups, confusion and hindrance of the medical staff no longer arises. The distance and the position between the patient and the evaluation unit is limited only by the range of the radio link. In addition, the costs which very frequently arise due to faulty cables are avoided.

The explicit association between transmitter and receiver and in the case of multichannel measurement paths also the latter's explicit association between the respective measuring device, such as a blood pressure sensor, and the corresponding reception channel of the monitoring unit, such as a monitor, avoid mix-ups which otherwise arise as a result of incorrect cabling. In this case, operation and construction are significantly simplified. In addition, the connection operates without disturbances and securely, but is automatically dropped upon leaving the transmission area and is not automatically picked up again. It is also possible for the user to drop the connection at any time. Despite the use of electromagnetic (radio) waves, the extremely low transmission power means that there is no risk to the patients or to the staff, and other electronic equipment is not disturbed either.

Other features which are considered as characteristic for the invention are set forth in the appended claims.

Although the invention is illustrated and described herein as embodied in a patient monitoring apparatus, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 is a schematic and block circuit diagram showing a basic structure of an apparatus for patient monitoring;

FIG. 2 is a block circuit diagram of the patient monitoring device; and

FIG. 3 is a flowchart of a functional sequence of connection setup and data transmission.

### DESCRIPTION OF THE PREFERRED EMBODIMENTS

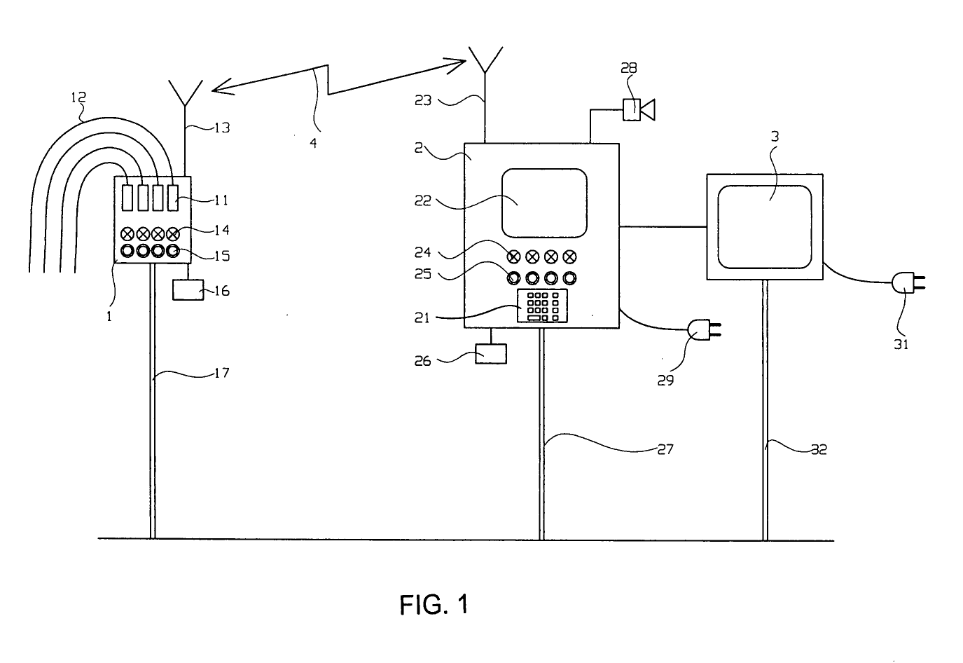

Referring now to the figures of the drawings in detail and first, particularly to FIG. 1 thereof, there is seen a basic structure of the patient monitoring apparatus. The patient monitoring apparatus has a transmission unit or transmitter 1, a reception unit or receiver 2 and an evaluation unit 3 which is connected to the reception unit 2. The transmission unit 1 and the reception unit 2 are connected to one another through the use of a 2-way radio link 4. In this context, 2-way means that data can be transmitted both from the transmission unit 1 to the reception unit 2 and from the reception unit 2 to the transmission unit 1. In this case, the terms transmission unit and reception unit are chosen such that they relate to the transmission of measured data.

The transmission unit 1 has one or more transducers 11 fitted thereto which are connected to a patient through the use of connecting lines 12. The transducers 11 may, by way of example, be pressure transducers which are connected to a patient's blood vessel, such as a vein or artery, through the use of the lines 12. These pressure transducers convert a fluid pressure into an electrical signal which, as described further below, is transmitted from the transmission unit 1 via an antenna 13 (which is fitted in a housing or on a board and cannot be seen from the outside) and the radio link 4 to an antenna 23 on the reception unit 2, after data processing.

As is known per se, the transducers 11 may preferably be fixed on the transmission unit 1 and electrically coupled thereto by plugging them on. This can be done by using inherently known electrical contacts on the transducers 11 and mating contacts on the transmission unit 1 or else by using short electrical cables.

The transmission unit 1 has a plurality of indicator lights 14, which are preferably in the form of light-emitting diodes (LEDs), as well as a plurality of keys 15, which will be discussed further below. The transmission unit 1 has a voltage supply 16 which is preferably in the form of a chargeable storage battery. The transmission unit 1 is fitted to a support 17, which may be a support rod or any other fixing apparatus that can be used to fix or set up the transmission unit close to the patient, for example including on the patient's bed.

Signals transmitted over the radio link 4 are transmitted on the basis of the “Bluetooth protocol”, which was explained at the outset herein. In this context, the range is approximately 10 m and requires no direct “line of sight” between the two antennas 13 and 23. The antennas therefore also do not need to be oriented to one another. Rather, they radiate and receive omni-directionally.

The reception unit 2 has an input device 21 fitted thereto, which in the simplest case may be a numerical keypad, a bar code reader, a magnetic card reader or any device for inputting a PIN. The input device 21 may further be an extended alphanumeric keypad. In addition, a display 22 is fitted on the reception unit 2. Furthermore, one or more light-emitting diodes 24 and a plurality of electrical keys 25 are provided. The power supply for the reception unit may be provided through the use of an external storage battery 26 and/or through the use of a mains cable 29. When power is supplied over the mains, the storage battery 26 serves as a buffer for emergency power supply.

The reception unit 2 can be fixed on a support 27, such as a support rod. The reception unit 2 may alternatively be fixed on a wall or may be set up on a table, since it is normally stationary. In addition, the reception unit has an audible signal transmitter 28 which produces an audible warning signal for particular operating states.

The reception unit 2 is connected to the evaluation or monitoring unit 3, which may be a conventional monitor or else a more complex device, such as a computer which logs all transmitted patient data. The evaluation unit 3 is normally supplied with power from the electrical mains through the use of a mains cable 31. The evaluation unit 3 may be fixed on a support 32, which is a recording desk, for example.

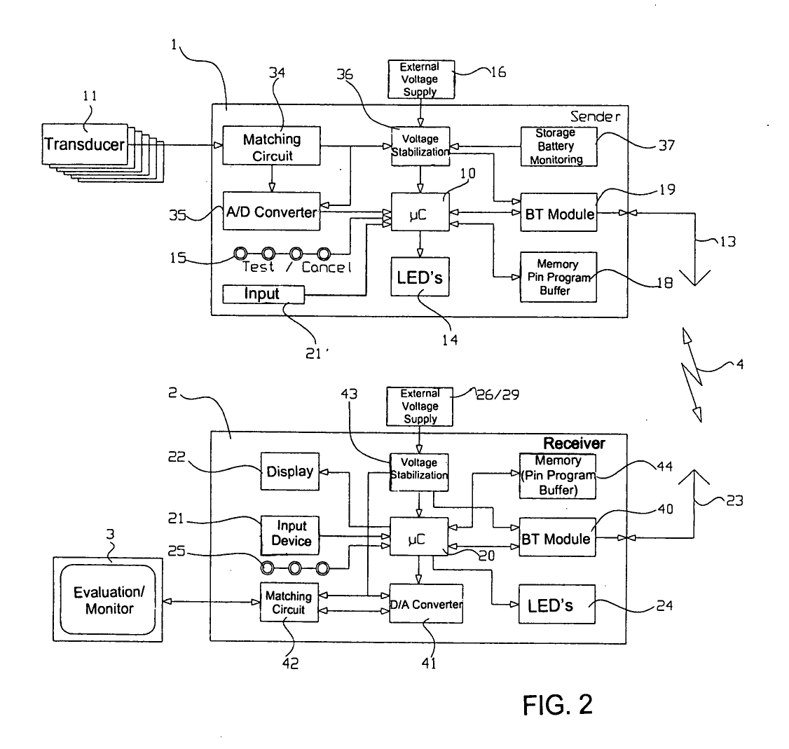

FIG. 2 shows a block diagram of the transmission unit 1 and the reception unit 2. The transmission unit 1 has one or more transducers 11 connected thereto. The core piece of the transmission unit 1 is a microcontroller 10, which receives electrical signals from the transducers 11 over a matching circuit 34 and an analog/digital converter 35. An input in the form of an input device 21′ is connected to the microcontroller 10. The microcontroller 10 has an associated memory 18 which, inter alia, contains an explicit identification code (subsequently referred to as a PIN) that is allocated to each transmission unit 1 individually and in such a way that it cannot be mixed up. In addition, the memory 18 stores a program for the flow control in the microcontroller 10. Finally, the memory 18 is also used as a data buffer, which is explained in more detail further below.

A “Bluetooth module” 19, which is connected to the microcontroller 10, converts the measured data conditioned by the microcontroller 10 into the Bluetooth protocol and sends them as radio signals over the radio link 4 using the antenna 13.

The LEDs 14 as well as the keys 15, which trigger “Test” and “Drop Connection” (cancel) functions, for example, are also connected to the microcontroller 10.

The external voltage supply 16 is connected to a voltage stabilization unit 36, which for its part supplies the microcontroller 10, the Bluetooth module 19, the matching circuit 34 and the A/D converter 35 with a stabilized voltage. A storage battery monitoring unit 37, which is also connected to the voltage stabilization unit 36, monitors the charge state of the storage battery 16 and reports it to the microcontroller 10 regularly through the voltage stabilization unit 36.

The direction of signal flow between the individual assemblies or functional parts is identified by respective arrows.

The core piece of the reception unit 2 is likewise a microcontroller 20, which is connected to a receiver-end Bluetooth module 40 which, for its part, is connected to the radio link 4 through the antenna 23. In this case too, a memory 44 is connected to the microcontroller 20 and likewise contains a program, serves as a buffer memory and also contains an identification code (PIN). The PIN code of the reception unit 2 can be changed as desired and can be input using the input device 21 which is connected to the microcontroller 20. The same PIN as that for the transmission unit 1 needs to be input on the receiver so that a radio link can be set up between the transmitter and the receiver. It is only then that a connection setup can be started, which is explained in more detail further below. The aforementioned LEDs 24, keys 25 and display 22 are also connected to the microcontroller 20. In addition, a digital/analog converter 41, which is connected to the microcontroller 20, is connected to the evaluation unit 3 through a matching circuit 42. Finally, in this case too, a voltage stabilization circuit 43 is provided which is connected to the external supply voltage, that is to say to the storage battery 26 and/or to the mains cable 29. In this case too, the respective direction of signal flow is identified by arrows. The connection between the evaluation unit 3 and the reception unit 2 may, as shown by the arrows, be two-way, which means that, by way of example, the evaluation unit 3 is also able to transmit signals, such as a data request or warning signals, to the reception unit 2.

It will be pointed out, in connection with the block diagram of FIG. 2, that the illustration shows functional units which do not necessarily have to be implemented as separate assemblies. By way of example, the A/D conversion and the D/A conversion can be carried out by the microprocessor. Likewise, the storage battery monitoring as well as the regulation of the voltage stabilization can be performed by the respective microcontroller 10 or 20 under program control.

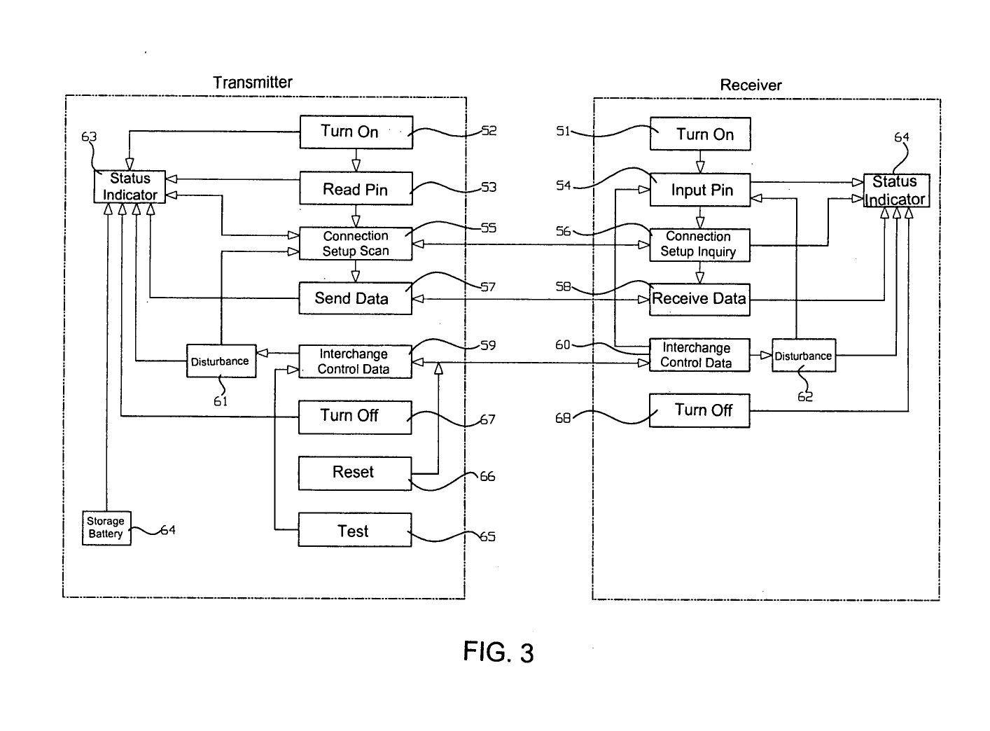

The manner in which the patient monitoring apparatus works will be explained in connection with FIG. 3. It is assumed that the transmission unit 1 and the reception unit 2 have been placed within the range of the radio link 4 and that both units are supplied with electrical power.

The receiver has been turned on, for example by turning on the power supply (block 51), and the microcontroller has been loaded with its program.

The transmitter has also been turned on (block 52), which may likewise be done by connecting the power supply, a switch or else by connecting at least one transducer 11. As mentioned, the transmission unit's memory contains an explicit identification code (PIN) which is read after the transmitter is turned on (Block 53). This code, which is preferably in the form of a numerical code, is attached to the transmission unit in such a way that it is clearly visible. This code needs to be input on the reception unit manually using the input device 21 (Block 54). It is only when this has been done that a connection setup between the transmitter and the receiver is started. During connection setup (Block 55), the transmitter is in the passive role as receiver and waits to see if a connection request is sent thereto.

This has the advantage that the transmitter consumes less power in this phase, causes no disturbances in the frequency band and is not actively transmitting. The active role during connection setup is performed by the receiver (block 56). As soon as a PIN has been input thereon, it starts to search for remote stations in its range, which is performed by using an “enquiry signal”. The Bluetooth protocol's usual “pairing” then takes place as soon as an activated transmitter (operating as a receiver at this time) has been found. The reciprocal PINs are then exchanged in order to set up the connection. If the two PINs from the transmitter and the receiver match, the connection is set up. If the PINs do not match, both parties return to the previous role, i.e. the transmitter will passively “listen” and the receiver will actively search, since an incorrect but likewise activated transmitter may have been contacted.

Once a connection has been set up successfully, further connection setup to other transmitters or receivers is disabled.

Following successful connection setup, useful data are transmitted by the transmitter (block 57) and are received by the receiver (block 58). In addition, control data are interchanged (blocks 59 and 60). Control data may include disturbance reports (blocks 61 and 62), for example, which are displayed on the transmitter and/or receiver as a status indicator (blocks 63 and 64) on the respective LEDs (14 and 24 in FIG. 2). It is also possible for normal operating states, such as storage battery OK (block 64), transducer connected, radio link present etc., to be indicated there. Pressing one of the keys 15 on the transmitter (block 65) permits a test routine for the respective transducer to also be able to be executed, for example by triggering a defined nominal value signal (100 mm/Hg is usual) in a known manner in the case of a pressure transducer. The nominal value signal is evaluated in the receiver 2 and is indicated on one of the LEDs 24 and/or the monitor 3. This allows a direct connection to be checked.

One of the keys 15 on the transmitter may also have an associated RESET function (block 66). The keys 15 on the transmitter and the input device 21 or the keys 25 on the receiver may also have an associated turn-off function (blocks 67 and 68), which are used to terminate the radio link. When one of these keys has been pressed, a connection is not set up again until a PIN has again been input on the receiver.

In the event of a disturbance on the radio link, this is indicated on the status indicators (blocks 63 and 64) and specifically on the LEDs 14 and 24. In addition, an audible alarm (signal transmitter 28 in FIG. 1) can be triggered.

If the disturbance on the radio link lasts for less than a prescribed time, for example 10 seconds, a new automatic connection setup operation is started. If the disturbance lasts for longer than this time, then the transmitter and the receiver change to their initialization state and a new connection setup operation cannot be performed again until after a PIN has been input on the receiver again (block 54). This reduces the risk of mix-ups. By way of example, if the transmitter is removed from the reception range, for example by virtue of the patient being taken out of the operating theater, a new connection setup operation to the same or to a different transmitter may be performed only after the PIN has been input again.

Manual dropping of the connection can be initiated by pressing a turn-off key (blocks 67 and 68) or a RESET key (block 66), which erases the PIN in the receiver. The radio link is also dropped when all transducers 11 have been isolated from the transmitter.

The receiver checks the present state of the connection and the connected transmitter checks its storage battery state at all times, and these are shown through the use of LEDs and/or on the display 22. In addition, the display shows plain-text messages, such as incorrect PIN, input PIN etc. When there is a connection to a transducer, status reports from this transducer may also be output in plain text. In practice, it is sufficient for the transmitter end to indicate the storage battery state on the LEDs and, when a plurality of transducers are being used, the ready status of the respective measurement channels. The storage battery state should expediently also be signaled audibly in addition, for a predefined critical state, e.g. using an intermittent or periodic alarm tone.

The LEDs may also indicate status using multicolored LEDs, for example using the colors red and green, with states additionally being able to be indicated through flashing. Expediently, the status indicators on the transmitter and receiver are identically constructed so as not to confuse the user with different indicators. The following states are signaled by the LEDs:
boot process, ready mode, connection setup, connection setup not possible, data transfer and storage battery OK, data transfer and storage battery critical, temporary connection disturbance, connection dropped as a result of disturbance, no connection, storage battery state low.

In addition, the transmitter may have further LEDs fitted thereon in line with the number of transducers which can be connected. It is possible to indicate whether the appropriate transducer is connected or not connected by turning the associated LED on or off.

The statements below may additionally be made in plain text on the receiver's display, with an input device 21 on the receiver also being able to be used to select a language (e.g. German, English, French, etc.):
boot process, ready for operation, input PIN, searching for connection, receiving data, information on the connection status, connection disturbances, connection dropped, storage battery state low, problem with connection setup, no remote station found.

The audible signal transmitter 28 on the receiver and/or on the transmitter may be a buzzer which generates a warning tone when the storage battery state is critical.

For many applications, the input device 21 on the receiver will suffice as a numerical keypad with a few additional function keys. This input device is used to input the PIN, possibly to correct the PIN, to confirm the PIN and to configure the language.

The RESET or CANCEL key is preferably highlighted with a special marking and is disposed on the transmitter and receiver at the same or at a suitably highlighted position.

The PIN is preferably defined for each transmitter module. This PIN may also contain the name of the hospital in coded form, which means that a 5-digit PIN is normally sufficient for the number of items normally found even in large clinics. In line with one variant of the invention, the PIN permanently stored in the transmitter may also be additionally printed as a barcode on the transmitter's housing, in which case the receiver has a barcode reader as the input device for inputting the PIN. This avoids possible errors when inputting the PIN using a keypad. In accordance with one alternative, the input devices 21′ and 21 of the transmitter 1 and the receiver 2 may also be respective readers for a magnetic or chip card on which the PIN for the transmitter and receiver is stored and is respectively input before compiling a pair from the transmitter and receiver. Since the PIN is erased for a prescribed period (e.g. after 10 seconds) when the radio link is interrupted, there is also no risk of a transmitter which has just been brought into an operating theater, for example, still “storing” an “old” PIN which matches the PIN that is stored on the receiver.

Disturbances on the radio link are, in principle, identified by the Bluetooth system and normally prompt the radio link to be set up again. However, the invention provides for this to be done only for interruptions within a particular period. In the case of longer interruptions, it will not be possible to pick up again automatically.

The result of brief interruptions is that feeding in digital input values in the receiver's D/A converter gives rise to interruptions, which in turn results in the converter steps no longer being able to be equidistant. For this reason, the sensor data are buffered in the receiver's memory 44, which increases the stability of the data transfer. This stability is admittedly bought by increasing a delay time, which is greater the larger the volume of buffered data.

Since the data rate via the radio link (e.g. 721 kBit/s) is much higher than the required data rate for the transducer (approximately 9.6 k), the transmitter's Bluetooth module 19 can be disconnected in the interim in order to save energy. After the transmitter-end Bluetooth module 19 has been connected, all values obtained which have been buffer-stored, i.e. buffered, in the transmitter can be transmitted. The resultant delay is non-critical in this case and can be fixed at appropriate values of up to a few seconds, for example.

When measuring blood pressure, the measured values from the transducers are converted at a sampling rate of 200 Hz and at a resolution of 12 bits, which gives a data rate of 2400 bit/s per measurement channel. The data to be transferred are transferred by the microcontroller in a prescribed transfer format packed as a “frame” and in packets. Each data packet can contain a sequence number which is continuously incremented. In addition to the digitally converted measured data, a data packet may also have control data inserted into it, for example about storage battery state, test button pressed, authentication request, authentication response, disturbance and identification of the respective transducer.

This application claims the priority, under 35 U.S.C. § 119, of German Patent Application 10 2004 012 042.0, filed Mar. 10, 2004; the entire disclosure of the prior application is herewith incorporated by reference.

1. A patient monitoring apparatus, comprising:
a reception unit, said reception unit having an input device for inputting an identification code, and said reception unit having a memory for storing the input identification code;
a transmission unit for a patient, said transmission unit storing a unique identification code, and said transmission unit transmitting measured data to said reception unit over a radio link;
at least one transducer for the patient, said at least one transducer being connected to said transmission unit; and
said transmission unit and said reception unit having a connection set up therebetween only if the identification codes in said transmission unit and said reception unit are identical.

2. The patient monitoring apparatus according to claim 1, wherein the identification code stored in said reception unit is erased following a disturbance on said radio link between said transmission unit and said reception unit for longer than a prescribed period, and a connection can be set up again only after the identification code has been input on said reception unit again.

3. The patient monitoring apparatus according to claim 1, wherein said transmission unit and said reception unit each have a respective Bluetooth module, and said radio link between said transmission unit and said reception unit is based on the Bluetooth protocol.

4. The patient monitoring apparatus according to claim 1, wherein said transmission unit and said reception unit each have a respective microprocessor and at least one respective memory connected to said respective microprocessor, and said microprocessors use programs stored in said memories to control a flow for setting up said radio link with said reception unit actively transmitting request signals and said transmission unit passively waiting for reception of request signals and changing to an active transmission mode only after said reception.

5. The patient monitoring apparatus according to claim 1, wherein said transmission unit has a memory buffer-storing data picked up by said at least one transducer and transmitted to said reception unit packet by packet at prescribed intervals of time.

6. The patient monitoring apparatus according to claim 3, wherein said transmission unit has a memory buffer-storing data picked up by said at least one transducer and transmitted to said reception unit packet by packet at prescribed intervals of time, and said Bluetooth module in said transmission unit is deactivated for transmissions in time intervals in which no data packets are being transmitted.

7. The patient monitoring apparatus according to claim 5, wherein the data packets contain control data in addition to the measured data.

8. The patient monitoring apparatus according to claim 7, wherein said transmission unit has a storage battery and a monitoring circuit monitoring a charge state of said storage battery and generating corresponding storage battery monitoring data being transmitted to said reception unit as control data.

9. The patient monitoring apparatus according to claim 1, wherein said transmission unit and said reception unit each have respective identical indicator lights for signaling prescribed operating states.

10. The patient monitoring apparatus according to claim 1, wherein said transmission unit and said reception unit each have at least one respective identical key for interrupting said radio link and erasing the stored identification code in said reception unit.

11. The patient monitoring apparatus according to claim 1, wherein the unique identification code in said transmission unit is firmly factory-prescribed, and said input device of said reception unit is a keypad.

12. The patient monitoring apparatus according to claim 1, wherein said transmission unit and said reception unit each have a respective input device for reading in the identification code from a common data storage medium.

13. The patient monitoring apparatus according to claim 12, wherein said common data storage medium is a magnetic card.

14. The patient monitoring apparatus according to claim 12, wherein said common data storage medium is a chip card.
